(19) United States
(12) Patent Application Publication
(10) Pub. No.: US 2005/0244910 A1
(43) Pub. Date: Nov. 3, 2005

(54) Methods for determining glycemic responses of foods

(76) Inventors: Thomas M.S. Wolever, Toronto (CA); Blanche Ip, Toronto (CA); Elham Mogghadam-Bozorgi, Burlington (CA)

Correspondence Address: KRAMER & AMADO, P.C., 1725 DUKE STREET, SUITE 440, ALEXANDRIA, VA, 22313, US

(21) Appl. No.: 11/118,441

(22) Filed: May 2, 2005

(30) Foreign Application Priority Data
Apr. 30, 2004 (CA) 2,465,655

(51) Int. Cl. C12Q 1/54

(52) U.S. Cl. 435/14

(57) ABSTRACT

The present invention provides precise methods for determining the glycemic responses of foods, including: (a) the incremental area under the glycemic response curve, (b) the Glycemic Index value of a food, (c) the Equivalent Glycemic Load or Glycemic Glucose Equivalent of a food, and (d) other similar measures.

### BACKGROUND OF THE INVENTION

The present invention relates to methods for determining the glycemic responses elicited by the consumption of foods. Foods elicit glycemic responses primarily due to their content of available carbohydrates.

Dietary Carbohydrates

Carbohydrates are polyhydroxy aldehydes, ketones, alcohols, acids, their simple derivatives and their polymers having linkages of the acetal type. Carbohydrates can be classified based on their chemical composition or their physiological effects.

The chemical classification of carbohydrates includes the number and nature of the monosaccharide units contained in the carbohydrate molecule. Monosaccharides are the basic building blocks of carbohydrates and usually contain 5 or 6 carbon atoms, 5 or 6 oxygen atoms and a number of hydrogen atoms. There are many types of monosaccharides including glucose, fructose, galactose, ribose, xylose and mannose. These monosaccharides can be reduced by the addition of hydrogen atoms to form sugar alcohols, such as sorbitol and xylitol, or compounds containing less than 5 carbon atoms such as erythritol. The same or different monosaccharides can be joined together to form long molecules, much like the links in a chain. Carbohydrate molecules containing one or 2 monosaccharide units are known as sugars, carbohydrates containing 3 to 9 monosaccharides are known as oligosaccharides, and carbohydrates containing 10 or more monosaccharides are known as polysaccharides.

The most abundant carbohydrates in the human diet include starch, a polysaccharide of glucose, the monosaccharides glucose (blood sugar) and fructose (fruit sugar) and the disaccharides sucrose (or table sugar, a disaccharide of glucose and fructose) and lactose (or milk sugar, a disaccharide of glucose and galactose). Hundreds of other types of naturally occurring carbohydrates are present in the human diet at low levels. Some of these naturally occurring carbohydrates are extracted and purified, or synthetically manufactured as food ingredients. In addition, novel carbohydrates can be synthesized and used as food ingredients.

Carbohydrates can also be classified according to their physiological effect. Carbohydrates differ in the rate and extent to which they are digested and absorbed in the human small intestine and the extent to which they are metabolized and retained in the body. Digestion refers to the breaking down of disaccharides, oligosaccharides and polysaccharides into their component monosaccharides, a process which occurs within the small intestine and/or at the surface of the cells lining the small intestine. Absorption refers to the process by which the monosaccharides are taken up by the cells lining the small intestine and either used for metabolic processes within those cells, or transported into the blood stream from which they can be carried to other parts of the body for use in metabolic processes. Metabolism refers to the use of the carbohydrates by body cells either as a fuel for energy or as building blocks for other compounds such as DNA. Sugars which are not metabolized are excreted in the urine.

About 75-80% of the carbohydrate absorbed by humans on a normal diet is glucose. Hence, the intestinal cells have an active transport system for glucose which means that glucose is rapidly and virtually completely absorbed. The absorption of other monosaccharides is not well understood, but is not as complete or rapid as that of glucose. Some non-glucose monosaccharides, such as mannitol, are poorly absorbed, and some, such as sorbitol, are partly absorbed, and some, such as fructose, are partly absorbed when consumed alone, but completely absorbed when consumed in the presence of glucose.

The digestion of disaccharides is mediated by enzymes (disaccharidases) on the surface of the cells lining the small intestine. Different enzymes are required for different sugars. Some disaccharidases, such as sucrase or maltase, are present in virtually all people, and, thus, virtually everybody can digest table sugar (sucrose) and maltose (the end product of starch digestion). Some disaccharidases, such as lactase which is needed to digest milk sugar, are either absent or present in low levels in a significant proportion of people. A low level of lactase activity may result in milk intolerance. Some dietary disaccharides, such as maltitol (a sugar alcohol), have no specific disaccharidase, but can be broken down at a slow rate by other disaccharidase. These types of disaccharides are only partly digested. Humans have no enzymes capable of digesting some disaccharides, such lactitol (a sugar alcohol), and these carbohydrates are completely indigestible.

The only human enzyme capable of digesting polysaccharides is amylase, present in low levels in saliva, but most abundantly in digestive juices secreted by the pancreas. Amylase acts within the lumen of the small intestine to break apart starch into short chains of glucose molecules which are finally digested to glucose itself by enzymes on the surface of the cells lining the small intestine. Amylase only digests starch; it does not act on other polysaccharides. Indigestible polysaccharides are commonly known as dietary fiber. The digestibility of starch varies depending on the nature of the starch molecules. Starch is classified as resistant starch, slowly available glucose, and rapidly available glucose. Resistant starch is not able to be digested in the human small intestine. Slowly available glucose refers to starch which slowly digested. Rapidly available glucose refers to starch which is rapidly digested. The overall extent of starch digestion depends on its rate of digestion; >95% of rapidly digested starch is absorbed from the small intestine. However, if the digestion of starch is slow, it may not be completely absorbed during its passage through the small intestine. Experiments suggest that 5-20% of the slowly digested starch in foods may actually escape digestion.

Dietary Carbohydrates and Glycemic Responses

Glycemic response refers to the change in blood glucose concentration which occurs after eating. Glucose is the major carbohydrate present in the human blood stream, and the major carbohydrate used as fuel by the body. Blood consists of a cellular and a liquid phase. The vast majority of the cells in blood are red cells, which carry oxygen around the body. There are also the so-called white cells, which are the cells of the immune system which fight infections. The liquid phase of the blood is called plasma. If a sample of blood is left to clot in a tube and then centrifuged, the liquid phase resulting is called serum. Serum differs from plasma in that the proteins involved in blood clotting are not present (having been left behind in the clot). Glucose is present in both the plasma and the red cells. The concentration of glucose in red cells is a little lower than that in plasma. Glucose can be measured in plasma, serum, red cells or whole blood. The term “glycemic response” or “blood glucose response” or the like used in this document refers to glucose measured in any of these compartments.

The magnitude of the glycemic response elicited by a carbohydrate-containing meal depends on 7 main factors: (a) the nature of the monosaccharide absorbed, (b) the amount of carbohydrate consumed, (c) the proportion of the consumed carbohydrate which is absorbed, (d) the rate at which the carbohydrate is absorbed, (e) the effects of other nutrients or components in the meal, (f) inter-individual variation and (g) intra-individual variation.

Glucose elicits a higher glycemic response, on a gram-for-gram basis, than any other monosaccharide. This is because the glucose consumed is the same molecule which appears as glucose in the blood. Other monosaccharides have to be converted to glucose by metabolic processes in the body if they are to appear as blood glucose. This process occurs at a slow rate, if at all. Thus, absorbed monosaccharides such as fructose, sorbitol, xylose or erithrytol have little or no effect on blood glucose. These sugars may enter into metabolic pathways (eg. fructose and sorbitol) and be able to be converted to glucose within cells, but this glucose does not appear in the blood in large amounts. Alternatively, some sugars may be absorbed, but partly (eg. xylose) or completely (eg. erithrytol) excreted in urine.

The glycemic response depends on the amount of carbohydrate consumed. The incremental area under the blood glucose response curve increases in a curvilinear fashion as the amount of carbohydrate consumed increases. A doubling of carbohydrate intake from 25 to 50 g results in a near doubling of the glycemic response, but an increase from 50 to 100 g results in only about a 35% increase in glycemic response.

In order to elicit a glycemic response, the carbohydrate has to be absorbed into the blood-stream. Thus, carbohydrates which are not digested or absorbed do not elicit a glycemic response. Such carbohydrates are termed “unavailable” or “indigestible” or “non-glycemic” carbohydrates. Carbohydrates which are absorbed and metabolized are termed “available” or “glycemic” carbohydrates.

The glycemic response elicited by a given amount of available carbohydrate is directly related to the rate at which it is absorbed. This has been shown in 3 ways: (a) strong correlations between the rate of digestion in vitro (ie. in a test tube) and the glycemic response in vivo; (b) slowly sipping glucose over a prolonged period of time reduces the glycemic response compared to taking the same amount in a single bolus; and (c) pharmacologic inhibition of the enzymes of digestion to an extent which does not cause appreciable carbohydrate malabsorption markedly reduces the glycemic response.

Other nutrients or components in the meal can influence the rate of gastric emptying (eg. fat), the rate of digestion and absorption (eg. viscous dietary fiber) or increase the rate of metabolism of glucose in the body (eg. protein by stimulating insulin secretion).

Glycemic responses vary in different individuals for many reasons related to the degree of insulin sensitivity, insulin secretion and glucose effectiveness in each person. People with high blood glucose have one of the various forms of diabetes mellitus, or impaired glucose tolerance or impaired fasting glucose. The glycemic response of individuals is commonly established by using an oral glucose tolerance test in which the glycemic response after consumption of 75 g glucose is measured.

Intra-individual variation refers to the fact that if the same subject consumes exactly the same test meal under standardized conditions on repeated occasions, the glycemic response varies. This will be discussed in more detail below.

Quantification of Glycemic Responses

The blood glucose response elicited by a meal consists of a pattern of changing blood glucose concentrations over a period of time, and, thus, requires blood glucose to be measured before the meal is consumed and at least one, but preferably more than one, point in time after the meal. A common method of assessing glycemic responses consists of measuring blood glucose in subjects in the morning after 10-14 hour overnight fasts before and at 15, 30, 45, 60, 90 and 120 minutes after starting to consume a test meal. This invention will be discussed in the context of this schedule of blood sampling, but would apply to any other schedule which might be used.

For ease of comparison it is useful to convert a series of blood glucose concentrations into a single value, and there are many ways this could be done, for example, the average of all the measurements, the difference between the starting value and the highest value (peak rise) or time at which the peak rise occurs, or the difference between the highest and lowest concentrations (maximum excursion). However, calculation of the area under the curve is used by most authors and is generally considered to be a very useful summary measure of the glycemic response. There are a number of ways of calculating the area under the curve; but for quantifying the extent to which foods raise blood glucose, it is generally considered that a measure of the incremental area under the glucose response curve is the preferred method. In this method, the blood glucose concentration before starting to eat is subtracted from the blood glucose concentrations measured after eating, and the area under the resulting curve is calculated. There are several different ways of calculating the incremental area under the curve, and the results obtained vary significantly depending on the method of calculation used and the schedule of blood sampling employed. This invention could be applied to any of these methods of calculating incremental area under the curve. However, most particularly it is applied to the method of calculating incremental area under the blood glucose response curve recommended by the FAO/WHO for determining the glycemic index value of foods, a method which has recently been shown to be more valid and precise than other methods. This method is termed incremental area under the blood glucose response curve (iAUC).

The iAUC describes the area under the blood glucose response curve and above the starting (baseline) concentration, ignoring any area beneath the baseline; The method of calculation of iAUC is described below:
For times t₀, t₁, . . . tₙ the blood glucose concentrations are G₀, G₁, . . . Gₙ, respectively:

$$iAUC = {\sum }_{n}^{x = 1} {A}_{x}$$

where Aₓ=the iAUC for the x^{th} time interval (ie. between tₓ₋₁ and tₓ).
For the first time interval (ie. x=1):
   if G₁>G₀, A₁=(G₁−G₀)×(t₁−t₀)/2
   otherwise, A₁=0
For the other time intervals (ie. x>1)
   if Gₓ≧G₀ and Gₓ₋₁≧G₀, Aₓ={[(Gₓ−G₀)/2]+(Gₓ₋₁−G₀)/2}×(tₓ−tₓ₋₁)
   if Gₓ>G₀ and Gₓ₋₁<G₀, Aₓ=[(Gₓ−G₀)²/(Gₓ−Gₓ₋₁)]×(tₓ−tₓ₋₁)/2
   if Gₓ<G₀ and Gₓ₋₁>G₀, Aₓ=[(Gₓ₋₁−G₀)²/(Gₓ₋₁−Gₓ)]×(tₓ−tₓ₋₁)/2
   if Gₓ≦G₀ and Gₓ₋₁≦G₀, Aₓ=0

While the iAUC is a scientifically valid way of expressing glycemic responses, it is not a practical way of classifying the glycemic responses of carbohydrate foods because iAUC differs markedly in different individuals. However, if the iAUC elicited by a food is indexed against the iAUC elicited by a reference food, then the resulting indexed value is generally considered to be valid for all individuals. There are some exceptions, for example the glycemic response elicited by milk is lower than normal in people with low intestinal lactase activity. However, these exceptions are relatively few, and the classification of carbohydrate foods based on their glycemic responses is considered to be practical and useful.

There are two general ways in which the glycemic responses of carbohydrate foods are classified; glycemic index (GI) and glycemic load (GL). The GI is the iAUC of a food expressed as a percentage of the iAUC after an amount of oral glucose equal to the amount of glycemic carbohydrate in the food. Thus, GI is a measure of the “quality” of the available carbohydrate in the food, and it is independent of the amount of food consumed. GL, on the other hand, is a measure of the extent to which a specific amount of food raises blood glucose. GL was originally defined as GI×g, where GI is glycemic index and g is the grams of available carbohydrate. However, there are various ways of expressing GL, and the term “g” could also mean grams of food or number of servings. Other analogous methods include glycemic glucose equivalent (GGE) which is GI expressed as a percentage of the GI of glucose; since the GI of glucose =100, GGE=GL. Another method is the Equivalent Glycemic Load (EGL), which is a measure of the amount of a standard food which would raise blood glucose to the same extent as the portion of test food. For example, the EGL of a low carbohydrate food bar might be ½ slice of bread—which means that the glycemic response elicited by one food bar is the same as that elicited by ½ slice of bread; ie. the iAUC elicited by the food bar=the iAUC elicited by ½ slice of bread. The precision of all these methods of classifying the glycemic responses of foods depends upon the intra-individual variability of the iAUC.

Intra-Individual Variation of iAUC and Glycemic Index (GI)

The iAUC elicited by a standard meal repeated on several occasions by the same individual under standardized conditions varies from day-to-day. Such iAUC values are normally distributed. Variation is conveniently expressed as the coefficient of variation (CV=100×mean/SD). The average CV for normal subjects who consumed 50 g available carbohydrate portions of white bread or glucose was approximately 25%. This means that about ⅔ of all iAUC values will be between 75 and 125% of the true mean value for an individual and ⅓ outside these limits; 95% of iAUC values will be between 50 and 150% of the true mean, and 5% will be outside of these limits. Thus, if iAUC is measured in a group of 10 normal subjects there is a 50% chance that one subject's response will be more than 50% greater or less than his or her true mean response. The average intra-individual CV for subjects with type 2 diabetes is about 15% compared to about 29% for subjects with type 1 diabetes.

The reasons for intra-individual variation of glycemic responses include subject factors and analytical variation. Subject factors may include numerous things such as day-to-day variation in diet and exercise, time of day of the test, time since the last meal, smoking, illness, etc. Variation in these factors may influence oral glucose tolerance quite considerably. For example, altering the type of carbohydrate consumed at dinner affected the blood glucose response elicited by a standard breakfast meal by 20-25%. In addition, it is known that blood glucose concentrations fluctuate somewhat on a minute-by-minute basis due to the fact that insulin is secreted in a pulsitile fashion. The CV of minute-to-minute blood glucose variations is on the order of about 5%. By contrast, analytical variability is very small, with a CV of about 1-2%. Analytical variation is the variation in the actual analysis of the blood glucose concentration, and since it is so small, usually the blood glucose analysis is only performed once on each sample of blood. Since analytical variation is so small, it would be expected that analytical variation contributes much less to intra-individual variation of glycemic responses than variation of subject's diet and activities or minute-to-minute fluctuations in blood glucose.

GI is the iAUC elicited by a test food expressed as a percentage of the iAUC elicited by a reference food (glucose or white bread) taken by the same subject. The average of these values in a group of subjects is the GI of the food. It has been shown that iAUC values differ in different subjects, but that the GI controls for these differences—ie. there is no relationship between the iAUC of a subject's response to oral glucose and the GI values obtained for foods in that subject. Thus, most of the variation of GI values is actually due to intra-individual variation of IAUC. Since the GI is the ratio of 2 independently variable values (numerator and denominator), the variation of the resulting ratio is greater than the variability of the numerator and denominator. Reducing intra-individual variation of iAUC will reduce the variation of GI values. For example, subjects with type 1 diabetes have greater intra-individual variation of iAUC values than subjects with type 2 diabetes (CV=29% vs 15%). The variation of GI values is also greater in subjects with type 1 diabetes than in those with type 2 diabetes (pooled SD for 19 foods, 27 vs 19).

Another way to improve the reliability of the estimate of iAUC is to use an average of repeated measures of iAUC elicited by the same test meal in the same subject. If SD is the variation of a single estimate of iAUC, then the variation of the mean of n determinations of iAUC is equal to SD/√n. Using the average of 3 determinations of iAUC elicited by the reference food has been shown to reduce variability of GI values and to make the distribution of the resulting GI values more normal. Many biological measures are normally distributed, by which it is meant that if the frequency of occurrence of values is plotted, the resulting graph is a bell-shaped curve with the peak (ie. highest frequency) being at the mean and with the values being symmetrically distributed around the mean. Individual subject GI values calculated from a single determination of iAUC elicited by the test food and the reference food form a skewed distribution, ie. the distribution curve is not symmetrical about the mean, but, in the case of GI, there is a long tail to the left, with the occurrence of a few extremely high values. Using the mean of 3 measures of iAUC in each subject to calculate the GI normalizes the distribution of the GI values.

It would be desirable to find other ways of reducing intra-individual variation in glycemic responses because this would be expected to improve the precision of estimates of the glycemic impact of foods and/or improve the statistical power of studies to detect differences in glycemic response between treatments. However, the costs and the benefits of such methods need to be considered before they can be recommended. Reducing intra-individual variation would reduce the cost of doing glycemic response tests because a given degree of precision and/or statistical power could be achieved with fewer subjects. However, this would only be so if it did not cost very much to reduce intra-individual variation, or if the reduction in variation achieved was large. Repeating tests, as has been described above, is an expensive way of reducing variation, but, repeating the test of the reference food has an important effect, and thus, the cost is justified. Other methods of reducing intra-individual variation might also add cost, such as having to provide subjects with a standard meal the night before tests. In addition, if subjects' activities were restricted (eg. no smoking, or no vigorous exercise allowed for 24 h before the test) this might reduce the willingness of subjects to participate in tests, and increase costs of recruitment and reduce the rate at which tests could be conducted.

It is generally assumed that controlling subjects' diet and activity before test days is beneficial to reduce variation. Methods such as not allowing smoking or vigorous exercise for 24 h and providing subjects with a standard meal for dinner the night before a test have been used in an attempt to improve the quality of test results. However, we showed recently that not allowing any smoking or vigorous physical activity before for 24 h, providing subjects with a standard dinner, and controlling the time of fasting to within ±15 minutes, paradoxically, tended to increase intra-individual variability of glycemic responses compared to restricting smoking only on the morning of the test, restricting only unusual vigorous activity, asking subjects to eat their normal dinner the night before and allowing the time of fasting to vary between 10 and 14 h.

Some investigators measure glucose in several fasting blood samples and use the average value as the baseline measure for glycemic responses, assuming, presumably, that this will improve the estimate of baseline glucose and, hence, reduce the variation of the glycemic response measures. However, the effect of this on the variability of iAUC is not known.

### SUMMARY OF THE INVENTION

This invention provides improved methods for determining the glycemic responses of foods than previously described.

The methods of this invention provide a systematic evaluation of the effects of reducing the variation of the estimate of fasting glucose and controlling the timing of the fasting blood sample on the variation of glycemic responses as determined by the iAUC and glycemic index. There are two ways to improve the precision of the estimate of fasting blood glucose, both of which involve using the average value of more than one measure of fasting glucose. One way is to measure glucose in 2 or more different fasting blood samples and take the average of the readings; this method reduces minute-to-minute variation in blood glucose. The other way is to analyze glucose 2 or more times in a single blood sample and take the average of the readings; this method reduces analytical variation. The fasting blood sample can be taken immediately before starting to eat or at a time some minutes before starting to eat.

This invention demonstrates that the magnitude of analytical variation is very small (SD=0.0556 mmol/L; CV=1.3% at average fasting glucose of 4.26 mmol/L) but that reducing analytical variation by using the average of 2 determinations of glucose in a single fasting blood sample taken immediately before eating reduces day-to-day variation in iAUC, both in absolute terms, and when expressed as a percentage of total variance, and improves the accuracy and precision of glycemic index values and other measures of glycemic response.

This invention demonstrates that the difference between 2 determinations of fasting glucose concentration in a single blood sample taken immediately before eating is >0.2 mmol/L in about 6% of samples. If, in these cases, glucose is measured a third time and the average of the closest 2 values taken to represent the fasting glucose concentration, this results in a further small reduction in the variation of IAUC compared to taking the average of the first 2 determinations.

This invention demonstrates that the magnitude of minute-to-minute variation (SD=0.161 mmol/L, CV=3.8% at average fasting glucose of 4.26 mmol/L) is approximately 3 times that of analytical variation. Nevertheless, reducing minute-to-minute variation by taking the average of a single determination of glucose in 2 fasting blood samples, one taken about 5 minutes before starting to eat, and the other immediately before starting to eat, has no effect in reducing day-to-day variation in iAUC, increases the day-to-day variation in iAUC when expressed as the percentage of total variance, and has little or no effect on the accuracy or precision of glycemic index values.

This invention demonstrates that the variation in iAUC is greater if the fasting blood sample is obtained 5 minutes before starting to eat than if the fasting blood sample is obtained immediately before starting to eat.

The fact that small analytical errors in metabolites result in larger errors in values derived from calculations using the metabolites is well known, and is based on the statistical principle that variances are additive. For example, the anion gap is a value used in clinical medicine to help determine if the low blood pH of an individual is due to a metabolic (eg. diabetes or poisoning) or respiratory (eg. breathing) abnormality. The anion gap is calculated by subtracting the sum of the concentrations of chloride and bicarbonate ions in blood from the sum of the concentrations of sodium and potassium. The point here is that it is well known that the variability (error) in the anion gap is the sum of the variabilities (analytical errors) of the 4 measures used to calculate it (chloride, bicarbonate, sodium and potassium). Thus, it is not unexpected that reducing the variation of the measured values by a small amount has a larger effect on the variation of the calculated values. However, this principle has never been applied to the calculation of area under the blood glucose response curve. In addition, the fact that reducing analytical variation has a greater effect on variation of iAUC compared to reducing minute-to-minute variation is unexpected, because the reduction in analytical variation achieved by taking the average of 2 determinations of blood glucose is much smaller than the reduction in minute-to-minute variation achieved by taking the average blood glucose in 2 different blood samples. Analytical variation was found to be 1.3% and minute-to-minute variation was found to be 3.8%. Taking the average of 2 measures reduces variation by 1/√2; thus analytic variation is reduced from 1.3 to 0.9% (reduction of 0.4%), whereas minute-to-minute variation is reduced from 3.8 to 2.7% (reduction of 1.1%). Indeed, the current state of the art teaches that using the average of several different fasting blood samples taken at intervals over 20-30 minutes prior to eating is the preferred method of increasing the precision of the estimate of fasting glucose for use in determining incremental glycemic responses.

### DETAILED DESCRIPTION OF INVENTION

This invention provides a simple method to reduce the variation of iAUC which improves the accuracy and precision of estimates of the glycemic responses of foods.

The method involves taking the fasting blood sample immediately before eating, measuring the glucose concentration 2 times and using the average of the 2 measures as the fasting glucose concentration. If the 2 measures differ by >0.2 mmol/L (3.6 mg/dL), then glucose is measured a 3^{rd} time and the average of the 2 closest measures is used as the fasting glucose concentration. Taking the average of blood glucose measured in several fasting blood samples taken at intervals before eating is not an effective method of reducing iAUC variation, and taking the fasting blood sample several minutes before eating results in higher variation of iAUC than taking it immediately before eating.

Theoretical Considerations and Rationale

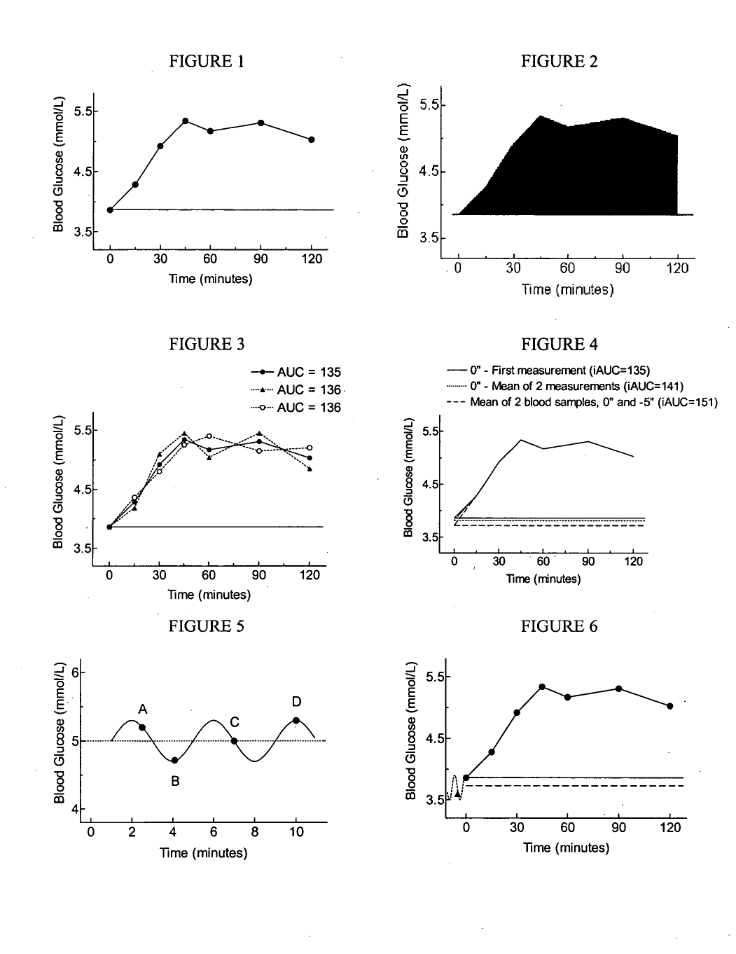

The method of calculating iAUC has been described above. FIG. 1 shows a sample glycemic response of a normal subject. The iAUC, 135 mmol×min/L, is the area above the fasting (time 0 min) glucose concentration and below the curve. This area is shaded in FIG. 2.

Normally glucose is only measured once in each blood sample. Since each measure of blood glucose is subject to both biological and analytical variation then the estimate of blood glucose actually measured could be higher or lower than the true value. If the variation is random, then some of the measured values will be greater than the true value and some less, and average of all the values would likely be very close to the true value. FIG. 3 illustrates this by showing 3 blood glucose curves; the solid line is the original curve as shown in FIG. 1, and the other 2 curves connect hypothetical small variations in blood glucose concentrations in each of the 6 blood samples taken after eating. Assuming the fasting glucose is the same in each case, it can be seen on FIG. 3 that random variations in the blood samples after eating have little or no effect on the iAUC computed.

FIG. 4 shows the same blood glucose curve as in FIGS. 1-3, but now with small variations in fasting glucose. Since the fasting glucose concentration is subtracted from every other value in order to calculate the iAUC, small random error in the estimate of fasting glucose concentration contribute to relatively large differences in iAUC. The solid horizontal line in FIG. 4 is the first estimate of fasting glucose measured in a blood sample taken immediately before eating, resulting in an iAUC of 135. The horizontal dotted line is the average of 2 determinations of fasting glucose concentration in the blood sample taken immediately before eating. The mean of the 2 determinations is only 0.05 mmol/L (1.3%) less than the first measurement, but this difference adds 0.05 mmol/L×120 min=6 mmol×min/L to the iAUC, which, in this case is 4.4% of the original value. The dashed line is the average of the blood glucose concentrations in a blood sample taken immediately before eating and another taken 5 minutes previously. The average is 0.14 mmol/L (3.6%) less than the original value, resulting in a 12% increase in iAUC. This illustrates how small differences in the estimate of fasting glucose can result in relatively large errors in iAUC.

Analytical Variation in Blood Glucose

Glucose concentration is usually analyzed by chemical reactions involving enzymes and substrates in which the enzyme reacts with the glucose to produce a product which is measured by a detector. The rate of production of the product depends on the concentration of enzymes, substrates and glucose, and factors such as the temperature at which the reaction is carried out, and subject to errors in the measurement of volumes and weights of reagents and blood included, as well as other factors such as temperature, mains electrical supply to the apparatus, etc. However, these errors are typically very small. The particular method of glucose analysis used for this invention is an automatic glucose analyzer (Model 2300 STAT, Yellow Springs Instruments) in which the entire analysis is automated and the error is very low, with CV's in the order of 1-2%. A CV of <3% is usually considered acceptable for medical laboratory analyses.

In example 1, 112 samples of fasting blood glucose were obtained from 14 normal subjects, and each sample was analyzed 3 times, and the mean and SD of the 3 glucose determinations (G₁, G₂ and G₃) in each sample were calculated as follows:
mean=(G₁+G₂+G₃)/3
SD=√([(G₁²+G₂²+G₃2)−{(G₁+G₂+G₃)²}/3]/2).

The overall mean glucose concentration in the 112 samples was 4.26 mmol/L (76.7 mg/dL) and the average of the 112 SD values was 0.0556, for a CV of 1.30%.

Minute-to-Minute Variation in Blood Glucose

Studies in which glucose has been measured at 1 minute intervals show the existence of approximately sinusoidal fluctuations with amplitude varying from about ±0.05 to 0.20 mmol/L (0.8 to 3.6 mg/dL) above or below the mean and frequency of peaks varying from 5 to 10 minutes. As an illustration of minute-to-minute variation in blood glucose, FIG. 5 shows sinusoidal fluctuations in blood glucose, with the sine wave varying about a mean of 5.0 mmol/L with an amplitude of 0.3 mmol/L and frequency of 4 minutes. The 4 dots along the line represent the variation in glucose which would be seen if blood samples were obtained at those moments in time.

To determine the magnitude of minute-to-minute variation in fasting glucose, 2 fasting blood samples were obtained at 5 min intervals from 14 normal subjects on 4 separate days. Blood glucose was determined 3 times in each sample, and the average of the 3 measurements used for the analysis. This resulted in 48 pairs of blood glucose determinations taken 5 minutes apart. The SD of the differences was calculated using the following formula:
SD=√[(ΣD²)/48]
where D=the difference in glucose concentration between the pair of blood samples taken 5 minutes apart. The resulting SD was 0.161 mmol/L (2.9 mg/dL); the overall mean fasting glucose as 4.26 mmol/L (76.7 mg/dL) resulting in a CV of 3.78%.

FIG. 6 shows a possible explanation for why reducing minute-to-minute variation by taking the average glucose concentration of 2 different blood samples does not reduce the variation of iAUC. This figure represents the minute-to-minute fluctuations of fasting glucose as the dotted sine wave to the left of 0 minutes on the x-axis. The points and solid line show the blood glucose response elicited by a test meal and the baseline value. The area above the baseline and below the curve is the iAUC. Note that blood glucose starts to rise from the time that consumption of the test meal starts, ie. time 0 minutes. The blood glucose concentration at −5 minutes is represented by the solid triangle, and it can be seen that this is not the point from which blood glucose starts to rise after eating. Thus, the average of the blood glucose concentrations at −5 min and 0 min as the baseline (the dashed line) is not an accurate measure of when the blood glucose started rising, and, therefore, using this value to calculate the iAUC will result in increased error, which will be manifested as an increase in the day-to-day variation of iAUC.

### EXAMPLE #1

### Intra-Individual Variation of IAUC

Fourteen (14) normal subjects (Table 1) were studied on 4 occasions after overnight fasts. They consumed 4 different test meals consisting of either: 50 g glucose, 50 g glucose plus 10 g protein from soy protein concentrate plus 10 g fat from corn oil, a 50 g available carbohydrate portion of white bread, or a 50 g available portion of white bread plus 10 g protein from low fat cottage cheese plus 10 g fat from margarine.

Blood samples (2-3 drops) were taken by finger-stick. On each occasion, 2 fasting blood samples were taken separated by a 5 minute interval; these samples are termed −5 min and 0 min. As soon as possible after the second fasting blood sample, the subject started to eat one of the test meals and further blood samples were obtained 15, 30, 45, 60, 90 and 120 minutes after starting to eat. Glucose was analyzed using an automatic analyzer (Model 2300 STAT, Yellow Springs Instruments, Yellow Springs, Wis.). Blood glucose was measured 3 times in each fasting blood sample and once in each of the samples taken after eating. iAUC was calculated as described above using 9 different estimates of fasting blood glucose as follows:
FBG1: first analysis of glucose in 0 min sample (usual practice)
FBG2: average of first 2 analyses of glucose in 0 min sample
FBG3: average of all 3 analysis of glucose in 0 min sample
FBG4: average of first 2 (if within 0.2 mmol/L) or closest 2 measures of glucose in 0 min sample
FBG5: average of first measure of glucose in −5 min and 0 min samples
FBG6: average of all 6 measures of glucose
FBG7: first analysis of glucose in −5 min sample
FBG8: average of first 2 analyses of glucose in −5 min sample
FBG9: average of all 3 measures of glucose in −5 min sample

The iAUC values generated for each estimate of FBG were subjected to 2-way analysis of variance (ANOVA) examining for effects of test meal and subjects. An explanation of ANOVA follows along with an explanation of how this was used to determine the variation in iAUC values.

In this experiment, 56 values of iAUC will be generated, one for each test meal taken by each subject. These values all differ from each other because of potential differences between the subjects (main effect of subject) and differences between the test meals (main effect of test meal). The rest of the variation is considered to be due to random or day-to-day variation. In ANOVA, variation is calculated as the variance or sums of squares (SS). The assumption behind ANOVA is that the total variance (TSS) is comprised of the sum of the variance from the various sources of error; in this cases the sources of variation were considered to be subjects (SSS), meals (MSS) and random (or error) variation (ESS); ie.
TSS=SSS+MSS+ESS
Therefore: ESS=TSS−SSS−MSS

If there are I subjects (rows) and J test meals (columns) (in this case, I=14 subjects and J=4 test meals) of values, the value in the i^{th} row and j^{th} column is Aij, and ΣAi=the sum of the values in row i, ΣAj=the sum of values in column j and Σij=the sum of all values. Therefore:
TSS=Σ(Aij²)−(ΣAij)²/(IJ)
SSS=Σ[(ΣAi)²/J]−(ΣAij)²/(IJ)
MSS=Σ[(ΣAj)²/I]−(ΣAij)²/(IJ)

These values are calculated and an ANOVA table is created (Table 2) from which are calculated the Mean Squares (MS) and the F values. The F value for the main effect is the ratio of the ratio of the MS for the main effect divided by the error MS. A high F value means that the variation between the means is high compared to the random (day-to-day or error) variation. The F distribution can be used to assign a p-value, or the probability of obtaining such an F value by chance if the means really were not different from each other. A low p-value indicates that there is a low chance of the means being the same, or in other words, a high chance that the means really differ.

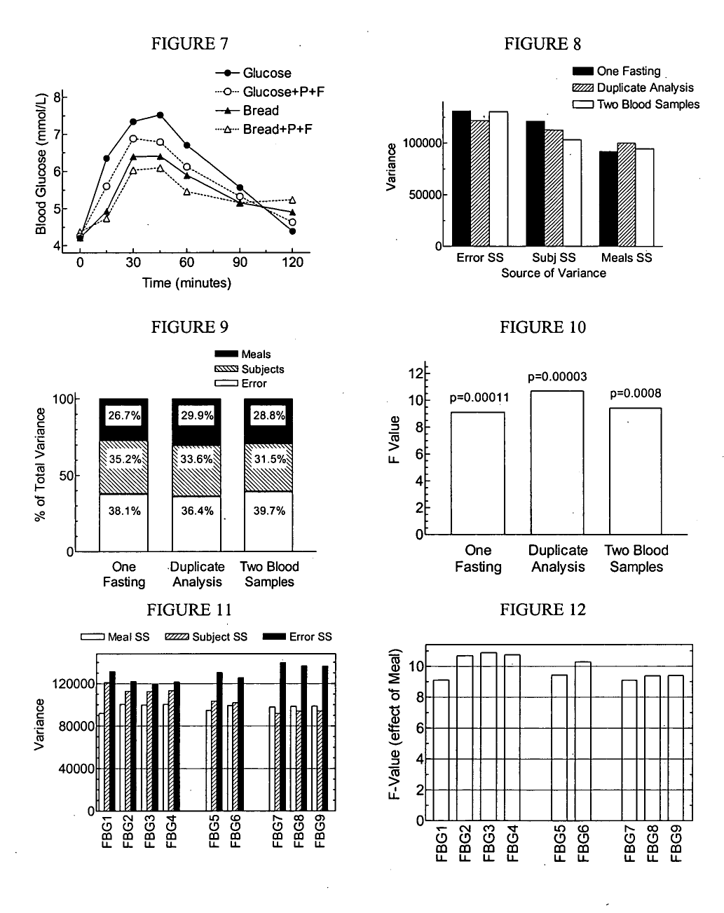

The blood glucose responses elicited by the 4 test meals are shown in FIG. 7. Whichever way FBG was calculated the main effect of test meal was highly significantly different.

A comparison of the results of the ANOVA of the iAUC values based on FBG1 (usual method of measuring glucose once in the 0 min sample—termed one fasting), FBG2 (average of 2 measures of glucose in the 0 min sample—termed duplicate analysis) and FBG5 (average of one measure of glucose in 2 blood samples—termed two blood samples) are shown in FIGS. 8 to 10. FIG. 8 shows that duplicate analysis reduced error SS, whereas two blood samples had no such effect. Also, subject SS was reduced by duplicate analysis and reduced even more by two blood samples, while test meal SS was increased to a greater extent by duplicate analysis than by two blood samples (FIG. 8). FIG. 9 shows that duplicate analysis reduced the day-to-day variation (error SS) when expressed as a percentage of total variation, while two blood samples actually increased the error SS when expressed as a percentage of total variation. FIG. 10 shows that duplicate analysis increased the F-value and reduced the p-value for the main effect of test meal to a much greater extent than did taking two blood samples.

FIG. 11 shows the partitioning of variance of iAUC values for all nine methods of determining FBG. It can be seen that the error SS (day-to-day variation of IAUC) is greater if FBG is taken at −5 min (FBG7) than at 0 min (FBG1), and that duplicate and triplicate analysis of glucose in the 0 min sample (FBG2 and FBG3) have a much greater effect in reducing error SS than doing duplicate and triplicate analysis of glucose in the −5 min sample (FBG8 and FBG9). Thus, error SS for the average of glucose in the 0 min and −5 min samples (FBF5) is intermediate between the duplicate determination in the 0 min (FBG2) and −5 min (FBG8) samples. Taking the average of all 6 measures of fasting glucose in both the 0 min and −5 min samples (FBG6) results in an error SS which is not as low as simply doing a duplicate analysis in the 0 min sample. Taking the average of triplicate analysis of glucose in the 0 min sample (FBG3) results in a slightly lower error MS than the average of duplicate analysis of glucose in the same sample (FBG2). Measuring glucose a third time only if the difference between the first 2 is >0.2 mmol/L (FBG4) results in an error MS intermediate between those of FBF2 and FBG3. Exactly the same can be said about the F-values derived from the different measures of fasting glucose (FIG. 12).

### EXAMPLE #2

### Precision of Estimate of Relative Glucose Response

The data from example #1 can by used to calculate the iAUC elicited by white bread as a percentage of that elicited by glucose. Each subject's iAUC after white bread alone was expressed as a percentage of the same subject's response after glucose alone, and the mean, SEM, CV and 95% confidence interval of the resulting values shown in Table 3. Compared to iAUC calculated from a single measure of glucose in the 0 min blood sample (FBG1), duplicate analysis of glucose in the 0 min sample (FBG2) reduced the SEM, CV and 95% confidence interval, and these values were not reduced any further by triplicate analysis of glucose in this blood sample (FBG3 and FBG4). By contrast, the precision of the estimate of white bread relative glycemic response was actually reduced (ie. higher SEM, CV and 95% confidence interval) by taking the average of blood glucose at −5 min and 0 min (FBG5), and was even worse when glucose in the −5 min blood sample was used to calculate IAUC (FBG7, FBG8 and FBG9).

### EXAMPLE #3

### Reducing Number of Subjects without Loss of Statistical Power

The data from example #1 can be used to show how duplicate analysis of fasting glucose allows for fewer subjects to be studied. Here, the F value for the main effect of test meal in 12 or 13 subjects is compared with the F value for all 14 subjects. The F value for all 14 subjects for iAUC values calculated using a single measure of glucose in the 0 min sample (usual method) was 9.11. Since there were 14 subjects, there are 14 different ways to obtain 13 subjects (removing each of the 14 subjects in turn, and calculating F for the remaining 13 subjects). When this is done for iAUC values calculated by the usual method, the resulting F value was less than 9.11 in 11 of the 14 (79%) cases. In other words, if only 13 subjects were used, there is about an 80% chance of obtaining a less significant result than using 14 subjects. However, if iAUC is calculated using the average of 2 measures of glucose in the 0 minute sample (new method), the F value in 13 subjects is less than 9.11 in only 3 of 14 cases (21%). In other words, there is an 80% chance of obtaining a more significant result with 13 subjects using the new method than with 14 subjects using the old method. The difference in these proportions, ie. 11/14 vs 3/14, is highly significant (p=0.002).

There are 91 different ways of reducing the number of subjects from 14 to 12. When iAUC is calculated using the usual method, the F value is less than 9.11 in 70 of the 91 cases (23%); ie. again there is about an 80% chance of losing statistical power in this set of subjects by reducing from 14 to 12 subjects. When iAUC is calculated using the new method, the F value is less than 9.1 in only 43 of 91 cases (47%; p=0.00004 compared to the usual method). In other words there is a little over a 50% chance of obtaining a more significant result with 12 subjects using the new method than with 14 subjects using the usual method.

In terms of a cost-benefit analysis, the effect of this invention depends on whether one wishes to improve the quality of the results or wishes to reduce costs. The cost of measuring glucose one extra time in the 0 min sample adds approximately 1% to the cost of doing a study. However; this resulted in an 11% reduction in the confidence interval of the relative glucose response (Table 3). This benefit may be relatively small in absolute terms; however, it is large compared to the very small cost of achieving it. Alternatively, the method described here allows a reduction in the number of subjects (and the cost of the study) to be reduced by 7% from 14 to 13 with a low risk of reducing statistical power. Thus, the invention described here could reduce the cost of determining glucose responses by about 6% without reducing the quality of the results.

TABLE 1
Subjects studied in Example 1

| | | Age | | Weight | Height | BMI |
|---|---|---|---|---|---|---|
| No. | Gender | (y) | Ethnicity | (kg) | (cm) | (kg/m²) |
| 1 | F | 35 | Persian | 67 | 166 | 24.3 |
| 2 | F | 23 | East Asian | 56 | 164 | 20.8 |
| 3 | M | 50 | Caucasian | 71 | 173 | 23.7 |
| 4 | M | 38 | Persian | 84 | 180 | 25.9 |
| 5 | F | 24 | East Asian | 50 | 165 | 18.4 |
| 6 | M | 19 | East Asian | 69 | 175 | 22.5 |
| 7 | M | 22 | Caucasian | 80 | 187 | 22.9 |
| 8 | F | 29 | African | 71 | 172 | 24.0 |
| 9 | F | 20 | Caucasian | 66 | 165 | 24.2 |
| 10 | F | 23 | East Asian | 52 | 164 | 19.3 |
| 11 | F | 19 | Caucasian | 70 | 173 | 23.4 |
| 12 | M | 22 | South Asian | 65 | 173 | 21.7 |
| 13 | F | 27 | South Asian | 54 | 153 | 23.1 |
| 14 | M | 27 | South Asian | 70 | 180 | 21.6 |

| | | | | |
|---|---|---|---|---|
| Mean ± SEM | 27 ± 2 | 66 ± 3 | 171 ± 2 | 22.6 ± 0.5 |

TABLE 2
2-way analysis of variance table with I subjects and J test meals.

| Source of Variation | df | Sum of Squares | Mean Square | F |
|---|---|---|---|---|
| Subjects | I − 1 | SSS | SMS = SSS/(I − 1) | SMS/EMS |
| Test Meals | J − 1 | MSS | MMS = MSS/(J − 1) | MMS/EMS |
| Residuals (Error) | (I − 1)(J − 1) | ESS | EMS = ESS/(I − 1)(J − 1) | |
| Total | IJ − 1 | TSS | | |

TABLE 3
Mean, SEM, coefficient of variation (CV) and 95% confidence
interval (CI) of the glycemic response elicited by white
bread expressed as a % of that elicited by oral glucose
in 14 normal subjects - comparison of different methods
of determining fasting blood glucose.

| Fasting Glucose* | Mean | SEM | CV | 95% CI |
|---|---|---|---|---|
| FBG1 | 73.2 | 6.7 | 34.1 | 13.1 |
| FBG2 | 69.2 | 5.9 | 32.0 | 11.6 |
| FBG3 | 69.0 | 6.1 | 32.9 | 11.9 |
| FBG4 | 69.3 | 5.9 | 32.1 | 11.6 |
| FBG5 | 72.1 | 6.8 | 35.2 | 13.3 |
| FBG6 | 69.5 | 6.5 | 35.1 | 12.8 |
| FBG7 | 71.2 | 7.3 | 38.6 | 14.4 |
| FBG8 | 70.3 | 7.4 | 39.3 | 14.5 |
| FBG9 | 70.4 | 7.4 | 39.5 | 14.6 |

*see text for definition of methods of determining fasting glucose.

1. Method for determining the glycemic responses of foods, the method comprising:
(a) selecting a food for testing;
(b) selecting subjects in whom glycemic responses will be measured;
(c) selecting how to take blood samples;
(d) selecting how to measure glucose in the blood;
(e) selecting the time when blood samples are taken; and
(f) selecting how the glycemic response is classified
and thereafter having the subjects ingest the food, taking the blood samples as selected, measuring the glucose in the blood as selected, at the selected times, and classifying the glycemic response.

2. A method according to claim 1, wherein the food is any dietary comestible or food ingredient tested either alone or added to another dietary comestible or food ingredient.

3. A method according to claim 2, wherein the amount of food tested is a uniform unit of the food.

4. A method according to claim 3, wherein the unit of food is based on the amount of carbohydrate it contains.

5. A method according to claim 3, wherein the unit of food is a portion containing 100 g or less carbohydrate.

6. A method according to claim 3, wherein the unit of food is a portion containing 50 g or less carbohydrate.

7. A method according to claim 3, wherein the unit of food is a portion containing less than 20 g carbohydrate.

8. A method according to claim 4, wherein carbohydrate is defined as total carbohydrate.

9. A method according to claim 4 wherein carbohydrate is defined as available carbohydrate, which means carbohydrate which is absorbed from the small intestine and metabolized in the body.

10. A method according to claim 9, wherein carbohydrate is defined as glycemic carbohydrate, which means total carbohydrate minus dietary fiber, minus other unavailable carbohydrates, minus the portion of sugar alcohols and partially absorbed and/or metabolized carbohydrates which are not absorbed and metabolized.

11. A method according to claim 3, wherein the unit of food is based on the energy it contains.

12. A method according to claim 3, wherein the unit of food is one or more servings.

13. A method according to claim 1, wherein the subjects are non-human.

14. A method according to claim 1, wherein the subjects are human subjects in any state of health.

15. A method according to claim 1, wherein the subjects are healthy subjects with normal or impaired glucose tolerance or type 2 diabetes mellitus.

16. A method according to claim 1, wherein blood samples are obtained by venepuncture from a forearm vein.

17. A method according to claim 1, wherein capillary blood samples are obtained by finger-stick.

18. A method according to claim 1, wherein glucose is measured in whole blood, plasma or serum.

19. A method according to claim 1, wherein glucose is measured using a hexokinase or glucose oxidase method.

20. A method according to claim 19, wherein glucose is measured using a hexokinase or glucose oxidase method using an automatic analyzer.

21. A method according to claim 1, wherein one fasting blood sample is taken within 2 minutes of starting to consume the food and any number of further blood samples are taken at intervals after eating.

22. A method according to claim 21, wherein blood samples after eating are taken at 15, 30, 45, 60, 90 and 120 minutes after starting to eat in subjects who do not have diabetes mellitus, and at 30, 60, 90, 120, 150 and 180 minutes after starting to eat in subjects with diabetes mellitus.

23. A method according to claim 21, wherein the analysis of glucose is repeated more than one time in the fasting blood sample.

24. A method according to claim 21, wherein the analysis of glucose is performed once in each of the blood samples after eating.

25. A method according to claim 21, wherein the analysis of glucose is performed more than once in each of the blood samples after eating.

26. A method according to claim 23, wherein the analysis of glucose in the fasting blood sample is repeated 2 times.

27. A method according to claim 26, wherein the analysis of glucose in the fasting blood sample is repeated 3 times if the second result differs from the first by more than 0.20 mmol/L (3.6 mg/dL).

28. A method according to claim 1, wherein the glycemic response is expressed as the area under a blood glucose response curve.

29. A method according to claim 28, wherein the area under the glucose response curve is calculated by subtracting the glucose concentration in the fasting blood sample from the glucose concentrations after eating and calculating the area under the curve from the resulting increments using the trapezoid rule.

30. A method according to claim 29, wherein an incremental area under the curve (iAUC) is calculated by including only the area above a baseline and ignoring the area below said baseline, wherein said baseline is the glucose concentration in the fasting blood sample.

31. A method according to claim 29, wherein a fasting glucose concentration is taken to be the average of the repeated measures of glucose according to claim 23.

32. A method according to claim 31, wherein if the results of the first 2 measures of fasting glucose differ by more than 0.20 mmol/L (3.6 mg/dL), then the baseline is taken to be the average of the 2 closest measures of fasting glucose.

33. A method according to claim 1, wherein the glycemic response is expressed as a glycemic index (GI).

34. A method according to claim 33, wherein GI is defined as the glycemic response elicited by a portion of a test food containing a known amount (X grams) of glycemic carbohydrate, according to claim 10, expressed as a percentage of the glycemic response elicited by X grams of a reference food in the same subjects, the method comprising:
(a) selecting subjects according to claim 13;
(b) taking blood samples according to claim 17;
(c) measuring glucose according to claim 19;
(d) calculating glycemic response as iAUC according to claim 30;
(e) testing the glycemic response elicited by the reference food 3 times in each subject and using the average of the iAUC in GI calculations; and
(f) calculating the GI of the food as the average GI in each of at least 6 subjects.

35. A method according to claim 34, wherein the portion of test food contains 50 g glycemic carbohydrate.

36. A method according to claim 34, wherein the blood samples are taken according to claim 22.

37. A method according to claim 34, wherein the reference food is anhydrous glucose.

38. A method according to claim 34, wherein the GI of the food is the average of at least 10 subjects.

39. A method according to claim 34, wherein the reference food is dextrose (glucose monohydrate).

40. A method according to claim 39, wherein the amount of dextrose used is 55 g.

41. A method according to claim 34, wherein the reference food is white bread.

42. A method according to claim 34, wherein the reference food is any starchy staple.

43. A method according to claim 1, wherein the glycemic response is expressed as an equivalent glycemic load (EGL).

44. A method according to claim 43, wherein EGL is defined as the amount of a standard comestible which would elicit the same glycemic response as a defined amount of a test food, the method comprising:
(a) any amount of test food can be used;
(b) selecting the subjects according to claim 13;
(c) taking blood samples by any method;
(d) measuring glucose by any method;
(e) calculating glycemic response as IAUC according to claim 30;
(f) determining the glycemic response elicited by 4 doses of the standard comestible in each subject and calculating a regression equation of iAUC on grams glycemic carbohydrate for each subject using the method of least squares, said equation expressed as: iAUC=Mg+B, where M and B are constants and g is the grams of glycemic carbohydrate in the standard comestible;
(g) calculating EGL for each subject as: EGL=(F−B)/M, where B and M are the constants and F is the iAUC elicited by the food; and
(h) calculating the EGL of the food as the average of the EGL values determined in at least 6 subjects.

45. A method according to claim 44, wherein a single serving of the test food is used and the food contains a low amount of glycemic carbohydrate.

46. A method according to claim 44, wherein blood samples are taken according to claim 17.

47. A method according to claim 44, wherein blood samples are taken according to claim 21.

48. A method according to claim 44, wherein EGL of the food is the average of the EGL values determined in at least 10 subjects.

49. A method according to claim 44, wherein the standard comestible is white bread, in which case EGL can also be termed bread equivalent (BE), which is the grams of glycemic carbohydrate from white bread which raises blood glucose to the same extent as the defined amount of test food.

50. A method according to claim 49, wherein the EGL is expressed in terms of the grams of white bread which raises blood glucose to the same extent as the defined amount of test food.

51. A method according to claim 49, wherein the EGL is expressed in terms of the number of slices of white bread which raises blood glucose to the same extent as the defined amount of test food.

52. A method according to claim 44, wherein the standard comestible is glucose, in which case ELG is the grams of glucose which raises blood glucose to the same extent as the defined amount of test food.

53. A method according to claim 44, wherein the standard comestible is any dietary comestible and EGL is expressed as grams of carbohydrate from said comestible or weight of said comestible or amount of energy from said comestible, or any other measure of said comestible.

54. A method according to claim 1, wherein glycemic response is expressed as relative glycemic response.

55. A method according to claim 54, wherein relative glycemic response is defined as the glycemic response elicited by a test comestible expressed as a percentage of the response elicited by a reference comestible, the method comprising:
(a) test and reference comestibles that can be any comestible food, ingredient or mixed meal, and the amounts thereof based on equal or unequal amounts of carbohydrate, fat, protein, energy or other nutrient or compound or ingredient contained therein, or based on their weight, number of servings, or any other way of classifying the amount;
(b) selecting the subjects according to claim 13;
(c) taking blood samples by any method;
(d) measuring glucose by any method;
(e) calculating glycemic response as iAUC according to claim 30.

56. A method according to claim 55, wherein blood samples are taken according to claim 17.

57. A method according to claim 55, wherein blood samples are taken according to claim 21.

58. A method according to claim 55, wherein glucose is measured according to claim 19.

59. A method according to claim 1, where glycemic response is expressed as glycemic load, glycemic glucose equivalent, or any other embodiment in which blood samples are taken according to claim 21, glucose measured according to claim 19 and which require calculation of area under the curve according to claim 29.

60. A computer usable medium for computing iAUC according claim 30.

61. A computer program product comprising computer usable medium having computer readable program code means embodied in said medium for determining iAUC according to claim 30.
